(19) United States
(12) Patent Application Publication
(10) Pub. No.: US 2011/0104139 A1
(43) Pub. Date: May 5, 2011

(54) ANGIOSTATIC COMPOSITIONS COMPRISING TRUNCATED TYROSYL-tRNA SYNTHETASE POLYPEPTIDES AND METHODS OF USING SAME

(75) Inventor: James E. Faber, Chapel Hill, NC (US)

(73) Assignee: THE UNIVERSITY OF NORTH CAROLINA AT CHAPEL HILL, Chapel Hill, NC (US)

(21) Appl. No.: 12/922,085

(22) PCT Filed: Mar. 11, 2009
(86) PCT No.: PCT/US09/36826

§ 371 Date: Dec. 15, 2010

Related U.S. Application Data
(60) Provisional application No. 61/069,062, filed on Mar. 11, 2008.

(51) Int. Cl. A61K 38/53; A61P 35/04; A61P 19/02; A61P 17/06; A61P 27/00

(52) U.S. Cl. 424/94.5

(57) ABSTRACT

Angiostatic compositions are provided comprising truncated forms of tyrosyl tRNA synthetase polypeptides. Also provided are methods of using such compositions in the treatment of conditions that benefit from decreased angiogenesis and/or neovascularization.

### CROSS-REFERENCE TO RELATED APPLICATIONS

This application claims the benefit under 35 U.S.C. §119(e) of U.S. Provisional Patent application No. 61/069,062 filed Mar. 11, 2008, which provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120161_{—}405PC_SEQUENCE_LISTING.txt. The text file is 9 KB, was created on Mar. 11, 2009, and is being submitted electronically via EFS-Web.

### BACKGROUND

1. Technical Field

The present invention relates generally to angiostatic compositions comprising truncated forms of tyrosyl tRNA synthetase polypeptides and methods of using such compositions in the treatment of conditions that benefit from decreased angiogenesis and/or neovascularization.

2. Description of the Related Art

Aminoacyl-tRNA synthetases, which catalyze the aminoacylation of tRNA molecules, are essential for decoding genetic information during the process of translation. In higher eukaryotes, aminoacyl-tRNA synthetases associate with other polypeptides to form supramolecular multienzyme complexes. Each of the eukaryotic tRNA synthetases consists of a core enzyme, which is closely related to the prokaryotic counterpart of the tRNA synthetase, and an additional domain that is appended to the amino-terminal or carboxyl-terminal end of the core enzyme. Human tyrosyl-tRNA synthetase (TyrRS), for example, has a carboxyl-terminal domain that is not part of prokaryotic and lower eukaryotic TyrRS molecules.

Mini-tyrosyl tRNA synthetase (mini-TyrRS), the N-terminal domain of TyrRS which corresponds to amino acid residues 1-364 and is cleaved by polymorphonuclear cell elastase and plasmin, is a member of the aminoacyl tRNA synthetase “ARS” multifunction cytokine-like proteins and peptides¹. In vitro, Mini-TyrRS has been shown to stimulate neutrophil activation and chemotaxis, endothelial cell proliferation and migration, and is pro-angiogenic in chick chorioallantoic membrane (CAM) and mouse matrigel assays¹⁻³. Mini-TyrRS has an ELR motif that, like CXC-chemokines such as IL-8, confers its chemokine and angiogenic activities. Like other ELR-containing cytokines, mutation of this motif inhibits mini-TyrRS binding and stimulation of leukoctyes and angiogenesis. Monocytes/macrophages, T-lymphocytes, and endothelial progenitor cells are important in angiogenesis, where they are recruited into and around new capillary sprouts and secrete growth factors and cytokines that promote endothelial cell proliferation and migration⁴.

Despite these in vitro observations, no studies have examined mini-TyrRS in physiological or pathophysiological settings in vivo. Therefore, we evaluated in vivo effects of mini-TyrRS on angiogenic processes. As described herein, it has been unexpectedly found that truncated TyrRS polypeptides exert biphasic effects on angiogenesis such that, at high doses, the polypeptides exhibit angiogenic properties, but at low doses, the polypeptides exhibit angiostatic properties.

### BRIEF SUMMARY

The present invention stems from the unexpected finding that low-dose in vivo administration of compositions comprising truncated tyrosyl tRNA synthetase (TyrRS) polypeptides inhibits angiogenesis, while high-dose administration of the same compositions augments angiogenesis.

Therefore, according to one aspect of the invention, there are provided pharmaceutical compositions and formulations which comprise a physiologically-acceptable excipient and an angiostatically-effective concentration of a truncated tyrosyl-tRNA synthetase polypeptide. The truncated tyrosyl-tRNA synthetase can comprise essentially any mammalian tyrosyl-tRNA synthetase, or active variant thereof, that is truncated at its C-terminus, and provides angiostatic effects when administered in accordance with the present invention. In certain embodiments, the truncated tyrosyl-tRNA synthetase comprises a tyrosyl-tRNA synthetase having at least 90% identity to the human tyrosyl-tRNA synthetase of SEQ ID NO: 1 and which is truncated at its C-terminus. In certain other embodiments, the truncated tyrosyl-tRNA synthetase comprises a human tyrosyl-tRNA synthetase of SEQ ID NO: 1 which is truncated at its C-terminus.

The extent of truncation at the C-terminus of a tyrosyl-tRNA synthetase can vary while still providing angiostatic effects. In certain embodiments of the invention, the truncated tyrosyl-tRNA synthetase has about 50-100 amino acid residues truncated from its C-terminus. In certain other embodiments, for example, the truncated tyrosyl-tRNA synthetase has about 100-150 amino acid residues truncated from its C-terminus. In certain other embodiments, the truncated tyrosyl-tRNA synthetase has about 150-200 residues truncated from its C-terminus. In certain other embodiments, the truncated tyrosyl-tRNA synthetase has about 200-250 amino acid residues truncated from its C-terminus.

In other embodiments of the invention, a truncated tyrosyl-tRNA synthetase of the invention comprises one or both of a Rossmann fold nucleotide binding domain and/or the sequence ELR.

In certain other embodiments, the truncated tyrosyl-tRNA synthetase consists essentially of amino acid residues 1-364 or 1-343 of SEQ ID NO: 1.

An angiostatically-effective concentration of a truncated tyrosyl-tRNA synthetase of the present invention may vary depending upon the particular route of administration and/or the condition being treated. In certain embodiments, the angiostatically-effective concentration is a concentration ranging from about 1-20 ug/kg 1-15 ug/kg, 1-10 ug/kg, 1-5 ug/kg, 5-10 ug/kg, 5-15 ug/kg or 5-20 ug/kg. Of course, it will be understood that these ranges may vary somewhat depending upon the indication to be treated, the mode of administration, etc., while still providing angiostatic activity according to the present disclosure and while still being within the spirit and scope of the invention.

According to another aspect of the invention, there are provided methods for treating a condition that would benefit from decreased angiogenesis comprising administering to a subject a composition comprising a physiologically-acceptable excipient and an angiostatically-effective concentration of a truncated tyrosyl-tRNA synthetase, as described herein. For example, in certain embodiments, the methods relate to the treatment of a condition selected from the group consisting of cancer (including solid and hematological tumors), rheumatoid arthritis, other arthritides, psoriasis, hyperangiogenic diseases, diabetic retinopathy, retinopathy of prematurity, ischemic retinopathy, macular degeneration, diabetic nephropathy, and sepsis.

### Brief Description of Sequence Identifiers

SEQ ID NO: 1 is the full length amino acid sequence of human tyrosyl-tRNA synthetase.

SEQ ID NO: 2 is a polynucleotide sequence encoding full length amino acid sequence of the human tyrosyl-tRNA synthetase of SEQ ID NO: 1.

### BRIEF DESCRIPTION OF THE SEVERAL VIEWS OF THE DRAWINGS

FIG. 1 illustrates the biphasic actions of Mini-TyrRS on recovery of blood flow in ear and hindlimb ischemia models. Top: Doppler perfusion was determined for the ear and hindpaw plantar surface in anatomically defined regions of interest (ROI) before and at indicated times after ligation of the central and peripheral ear arteries (“X” in first panel) or the femoral artery (last 5 panels); same mouse in each panel set. Pseudocolor bar spans 0-5,000 arbitrary perfusion units. A, B, Perfusion values normalized to non-ligated contralateral ear or paw. A, Recovery of ear perfusion was inhibited by low- and augmented by high-dose mini-TyrRS (mTyrRS), while “mutant” mini-TyrRS had no effect (“mutant” here and elsewhere is mini-TyrRS with the ELR motif mutated to EYR). Ear doses are the total daily dose from 2 injections given subcutaneously into base of ear (“*” in first panel) 12 hours apart. # p<0.05, ## p<0.01, ANOVA; **p<0.01, Bonferroni t-test. B, Recovery of hindlimb perfusion was inhibited by 12 ug/kg/h mini-TyrRS (*p<0.05, sc, osmotic minipump), in association with greater ischemic appearance scores (C). Values are mean±SEM for “n” number of animals for this and subsequent figures.

FIG. 2 illustrates the biphasic actions of Mini-TyrRS on capillary density seven days after ear artery ligation. Top, Representative 8-micron thick cross-sections of ear stained with anti-CD31 antibody. Bottom, angiogenesis was inhibited by low-dose and augmented by high-dose mini-TyrRS, while mutant mini-TyrRS had no effect compared with PBS. Fluorescence intensity was averaged for 8 fields spanning entire ear cross-section. #p<0.05, ##p<0.01, ###p<0.001 vs. sham ligation; *p<0.05, **p<0.01 vs. PBS. N=6-9/bar.

FIG. 3 illustrates that high-dose mini-TyrRS augments accumulation of CD45- and CD4-positive cells 7 days after ear artery ligation. Representative 8-micron thick sections of ear stained with anti-CD45 and anti-CD4 antibodies. CD45-positive cells are the average of 10 high power fields. CD4-positive cells are for entire ear cross-section. *p<0.01 vs. sham ligation, #p<0.001 vs. PBS. N=6-9/bar.

FIG. 4 illustrates that Mini-TyrRS has biphasic actions on macromolecular permeability in ear. A, mini-TyrRS, alone, at low-dose (sc) reduced and at high-dose augmented permeability, while mutant mini-TyrRS had no effect. B, Low-dose mini-TyrRS inhibited mustard oil (MO)-induced ear leakage, while high-dose or mutant mini-TyrRS had no effect. C, Low-dose mini-TyrRS (sc) inhibited VEGF-induced leakage in dorso-lateral trunk skin. D, Mini-TyrRS had biphasic actions on VEGF-induced leakage in endothelial cell monolayers. Trans-endothelial albumin flux expressed as percentage of clearance of Evans blue-conjugated bovine serum albumin, compared with untreated controls. $p<0.05, #p<0.01 vs. PBS, *p<0.05, **p<0.01, ***p<0.001 vs. VEGF or MO. N=4-7/bar.

FIG. 5 illustrates that Mini-TyrRS lacks vasoactive actions. A, Increase in perfusion (Doppler) induced in non-ligated ear by raising rectal temperature from 35° C. to 37.5° C. was unaffected by mini-TyrRS (20 μl subcutaneous administration into ear immediately after 35° C. measurement, followed by measurement 10 minutes later at 37.5° C.). B, Increase in perfusion induced by papavarine (adductor area sc) was unaffected by mini-TyrRS injected into same site 30 minutes earlier. C, Baseline (control) ear perfusion was unaffected after 6 days of daily sc mini-TyrRS (daily dose given as two 20 μl injections 12 hours apart). N=4-6/bar.

FIG. 6 illustrates that ischemia/hypoxia and VEGF reduce mini-TyrRS expression in vivo and in vitro. A and B, Western blot analysis of mini-TyrRS in gastrocnemius and adductor of sham-surgery mice, in gastrocnemius and adductor of leg with femoral artery ligation (lig), and in gastrocnemius of the contralateral non-ligated leg at the indicated days (d) after surgery. 20 ug (panels A,B) and 30 ug (panels C,D) protein per lane; normalized to tubulin. N=4/bar. C and D, Western blot analysis of mini-TyrRS in rat thoracic aorta maintained 4 days in organ culture with 100 ng/ml VEGF or 1% oxygen (hypoxia)±VEGF trap or IgG control (0.2 mg/ml). *p<0.05, **p<0.01 vs. sham or normoxia. N=3/bar.

### DETAILED DESCRIPTION

The present invention relates generally to the unexpected finding that low-dose in vivo administration of compositions comprising truncated tyrosyl tRNA synthetase (TyrRS) polypeptides inhibits angiogenesis, while high-dose administration of the same compositions augments angiogenesis. As further exemplified herein, N-terminal fragments of tyrosyl tRNA synthetase exhibited dose-dependent biphasic actions on ischemic angiogenesis and macromolecular permeability in vivo, i.e., anti-angiogenic, anti-permeability at low concentration and pro-angiogenic, pro-permeability at high concentrations, the latter in association with increased recruitment of CD4-positive T-cells.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

All publications, patents and patent applications cited herein are hereby incorporated by reference in their entirety.

As used in this specification and the appended claims, the singular forms “a,” “an” and “the” include plural references unless the content clearly dictates otherwise.

As used herein, the terms “polypeptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. Polypeptides of the invention may be prepared using any of a variety of well known recombinant and/or synthetic techniques, illustrative examples of which are further discussed below.

The present invention relates generally to compositions comprising an angiostatically-effective amount of a tyrosyl-tRNA synthetase polypeptide. Angiostatic activity provided by the compositions and methods described herein can be used to treat essentially any condition that would benefit from decreased angiogenesis. For example, angiostatic compositions of the invention may be used in treating or ameliorating the symptoms of disease conditions which rely upon angiogenesis and/or neovascularization, such as treating a solid tumor or tumor metastasis. The angiostatic compositions may also be used to treat conditions characterized by abnormal angiogenesis, such as rheumatoid arthritis, other arthritides, psoriasis, hyperangiogenic diseases, diabetic retinopathy, retinopathy of prematurity, ischemic retinopathy, macular degeneration, diabetic nephropathy. Further still, angiostatic compositions of the invention may be used to oppose the angiogenic activity of endogenous and/or exogenous angiogenic factors.

### Truncated Tyrosyl-tRNA Synthetase Polypeptides and Variants Thereof

As noted above, the compositions of the invention generally comprise one or more truncated tyrosyl-tRNA synthetase polypeptides. A “truncated tyrosyl-tRNA synthetase” or “truncated tyrosyl-tRNA synthetase polypeptide”, as used herein, refers to a tyrosyl-tRNA synthetase protein which has been truncated at its C-terminal end. The extent of the truncation, that is, the number of C-terminal residues removed from a full length tyrosyl-tRNA synthetase protein can vary while still providing desired angiostatic effects when compositions comprising the truncated polypeptide are administered in vivo, as described herein.

For example, in certain embodiments, at least about 5, 10, 15, 20, 25, 50, 75, 100, 150, 200, 250, 300, 350 amino acids, or more, including all intermediate lengths, are truncated from the C-terminus of a full length mammalian tyrosyl-tRNA synthetase, such as the full length human tyrosyl-tRNA synthetase protein sequence set forth in SEQ ID NO: 1. Intermediate lengths are intended to include all integers therebetween, for example, 6, 7, 8, etc., 51, 52, 53, etc., 201, 202, 203, etc.

In other embodiments, the truncated tRNA synthetase polypeptides is a C-terminal truncated form of SEQ ID NO: 1 which comprises a Rossmann fold nucleotide binding domain.

In other embodiments, the truncated tRNA synthetase polypeptides is a C-terminal truncated form of SEQ ID NO: 1 which comprises the sequence ELR.

In other embodiments, the truncated tyrosyl-tRNA synthetase is a carboxyl-terminal truncated form of SEQ ID NO: 1, which comprises a Rossmann fold nucleotide binding domain and further comprises the sequence ELR.

In other embodiments, the truncated tRNA synthetase polypeptides is a truncated tyrosyl-tRNA synthetase comprising amino acid residues 1-200, 1-250, 1-300, 1-350, 1-400, including all intermediate lengths, of SEQ ID NO: 1. Intermediate lengths are intended to include, for example, 1-201, 1-202, 1-203, etc., 1-250, 1-252, 1-253, etc., 1-301, 1-302, 1-303, etc.

In still other embodiments of the invention, the truncated tRNA synthetase polypeptides is a truncated tyrosyl-tRNA synthetase consisting essentially of amino acid residues 1-364 or 1-343 of SEQ ID NO: 1.

In another aspect, the present invention provides variants of the truncated tyrosyl-tRNA synthetase polypeptides described herein. Polypeptide variants encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to the corresponding region of a wild-type mammalian tyrosyl tRNA synthetase protein, such as SEQ ID NO: 1.

A polypeptide variant may differ from a naturally occurring tyrosyl-tRNA synthetase polypeptide in one or more substitutions, deletions, additions and/or insertions. Such variants may be naturally occurring or may be synthetically generated, for example, by modifying one or more of the above polypeptide sequences of the invention and evaluating their angiostatic activity as described herein using any of a number of techniques well known in the art.

In certain embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics, e.g., with angiostatic characteristics. When it is desired to alter the amino acid sequence of a polypeptide to create an equivalent, or even an improved, angiostatic variant of a tyrosyl-tRNA synthetase polypeptide of the invention, one skilled in the art, for example, can change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that generally defines that protein's biological functional activity, certain amino acid sequence substitutions can be made in a protein sequence, and, of course, its underlying DNA coding sequence, and nevertheless obtain a protein with like properties. It is thus contemplated that various changes may be made in the polypeptide sequences of the disclosed compositions, or corresponding DNA sequences which encode said polypeptides without appreciable loss of their angiostatic utility or activity.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). For example, it is known that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on secondary structure and hydropathic nature of the polypeptide.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Nat'l Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Nat'l Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Examples of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one illustrative approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

In certain embodiments of the invention, there are provided fusion polypeptides, and polynucleotides encoding fusion polypeptides. Fusion polypeptide and fusion proteins refer to a polypeptide of the invention that has been covalently linked, either directly or via an amino acid linker, to one or more heterologous polypeptide sequences (fusion partners). The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely effect the angiostatic activity of the polypeptide. For example, in one embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### Polynucleotide Compositions

The present invention also provides isolated polynucleotides that encode the truncated tyrosyl-tRNA synthetase polypeptides of the invention, as well as compositions comprising such polynucleotides.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a tyrosyl-tRNA synthetase or a portion thereof) or may comprise a variant, or a biological functional equivalent of such a sequence. Polynucleotide variants may contain one or more substitutions, additions, deletions and/or insertions, as further described below, preferably such that the angiostatic activity of the encoded polypeptide is not substantially diminished relative to the unmodified polypeptide. The effect on the angiostatic activity of the encoded polypeptide may generally be assessed as described herein.

In additional embodiments, the present invention provides isolated polynucleotides comprising various lengths of contiguous stretches of sequence identical to or complementary to a tyrosyl tRNA synthetase, wherein the isolated polynucleotides encode a truncated tyrosyl tRNA synthetase as described herein.

For example, polynucleotides are provided by this invention that encode at least about 100, 150, 200, 250, 300, 350, or 400, or more, more contiguous amino acid residues of a truncated tyrosyl-tRNA synthetase polypeptide of the invention, as well as all intermediate lengths. It will be readily understood that “intermediate lengths”, in this context, means any length between the quoted values, such as 101, 102, 103, etc.; 151, 152, 153, etc.; 201, 202, 203, etc.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides and fusions thereof may be prepared, manipulated and/or expressed using any of a variety of well established techniques known and available in the art. For example, polynucleotide sequences which encode polypeptides of the invention, or fusion proteins or functional equivalents thereof, may be used in recombinant DNA molecules to direct expression of a truncated tyrosyl-tRNA synthetase polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, expression and/or activity of the gene product.

In order to express a desired polypeptide, a nucleotide sequence encoding the polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of—galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as anthocyanins, -glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### Formulation and Administration

The compositions of the invention comprise a truncated tyrosyl-tRNA synthetase polypeptide formulated in pharmaceutically-acceptable or physiologically-acceptable solutions for administration to a cell, tissue or animal, either alone, or in combination with one or more other modalities of therapy. It will also be understood that, if desired, the compositions of the invention may be administered in combination with other agents as well, such as, e.g., other proteins or polypeptides or various pharmaceutically-active agents. There is virtually no limit to other components that may also be included in the compositions, provided that the additional agents do not adversely effect the angiostatic effects desired to be achieved.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain applications, the pharmaceutical compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the pharmaceutical compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Biphasic Effects of Truncated TyrRS Polypeptides on Angiogenesis

This examples demonstrates that low-dose in vivo administration of compositions comprising a truncated tyrosyl-tRNA synthetase polypeptide results in angiostatic effects, while high-dose administration of the same compositions results in angiogenic effects.

### Materials and Methods

a. Reagents.

Rabbit anti-mini-TyrRS antibody and human recombinant mini-TyrRS were from aTyr Pharma, Inc (La Jolla, Calif.). mFlt-trap (soluble VEGF-A receptor decoy) was kindly provided by Napoleon Ferrara and Stuart Bunting (Genentech). Bovine coronary venular endothelial cells were a gift from Cynthia Meininger, Texas A&M University. Four-to-five month-old mice were used in ear artery ligation (C57BL/6) and permeability models (sv129).

b. Unilateral Ear and Femoral Artery Ligation.

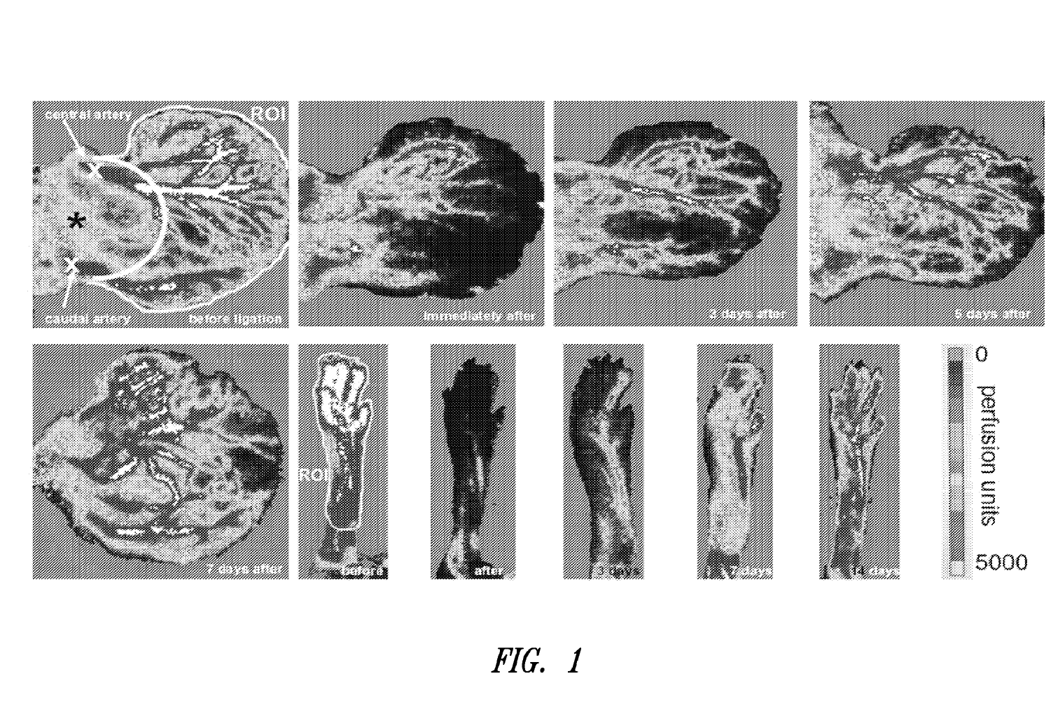

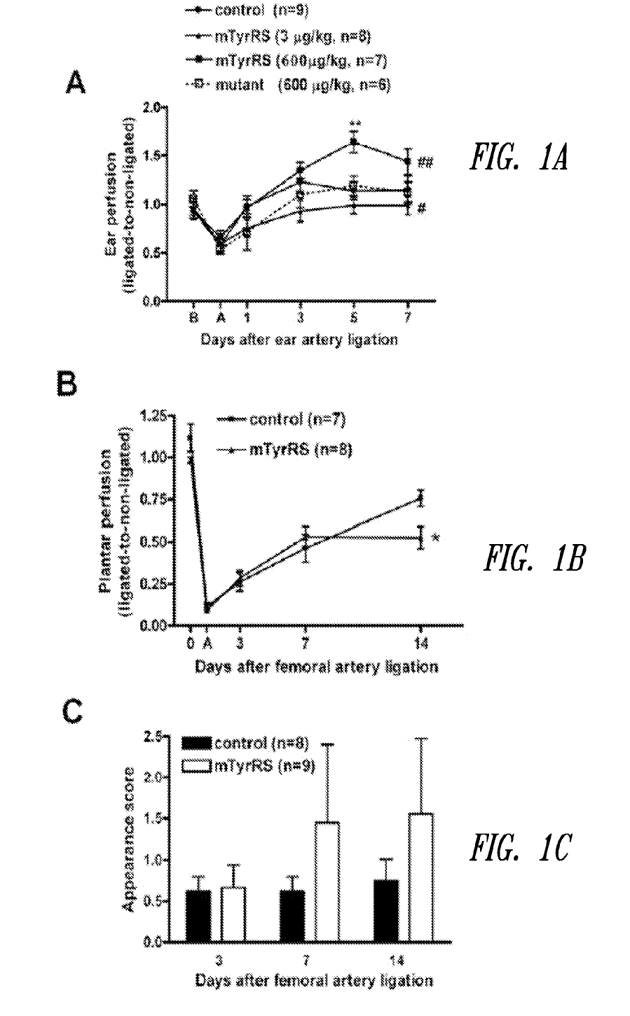

1-2 mm incisions were made overlying the central and peripheral ear artery trunks at their base in the pinna. Each artery was transected between two ligatures placed 1 mm apart⁵. The central ear artery was ligated distal to its lateral branch to prevent ear necrosis. 20 ul of phosphate buffered solution (PBS) containing mini-TyrRS, mutant mini-TyrRS or PBS (vehicle) was injected subcutaneously twice a day (FIG. 1). In other mice receiving mini-TyrRS or PBS by minipump, the right femoral artery was exposed through a 2-mm incision, carefully isolated from the femoral vein and nerve, ligated proximal to the genu artery and distal to the origin of the lateral caudal femoral and superficial epigastric arteries (the latter was also ligated), and resected between the ≈1 mm spaced ligatures⁶.

c. Laser Doppler Perfusion Imaging.

Animals were anesthetized with 1.125% isoflurane supplemented with 2:3 oxygen:air. Rectal temperature was maintained at 37° C. unless otherwise specified. Perfusion was obtained before ligation, immediately after, and on 1, 3, 5 and 7 days (ear model) and 3, 7 and 14 days (hindlimb model) using a scanning laser-Doppler perfusion imager (LDI2-IR, Moor Instruments) modified for high resolution⁶. Regions of interest (ROIs) were drawn using Moor software (FIG. 1). The ear ROI was defined as the region between the ear margin and a circle extending from a line drawn to connect the two pinna notches (FIG. 1). Unless otherwise indicated, ear perfusion was measured at 38° C. rectal temperature to minimize vasomotor tone and better indicate anatomic conductance.

d. Vascular Permeability.

Macromolecular permeability was measured in the ear or dorso-lateral back skin. Mini-TyrRS, mutant mini-TyrRS or PBS was injected (10 ul, 32 ga needle here and elsewhere) subcutaneously into the ear dorsum at the base of the pinna. Thirty minutes later 25 ul Evans blue dye (30 mg/kg) was administered via jugular vein. Immediately afterwards, allyl isothiocyanate (active ingredient in mustard oil; Sigma; diluted with mineral oil to 5% (v/v)), or mineral oil (control) was applied topically (5 ul) to the dorsal and ventral surfaces of both ears with a cotton-tip applicator. Thirty minutes later the vasculature was perfusion-fixed (1% paraformaldehyde (PFA) in 50 mM citrate buffer, pH 3.5) for 1 minute at 120 mmHg. Ears were removed, dried for 24 hours at 55° C. and weighed. Vascular leakage was indicated as Evans blue content extracted by incubation in 1 mL formamide for 48 hours at 55° C., and measured with a spectrophotometer at 610 nm against a standard curve⁷.

VEGF-induced permeability was examined in the shaved back skin. 20 ul PBS containing mini-TyrRS or PBS alone was injected subcutaneously. 30 minutes later Evans blue was injected IV as above, followed by VEGF-A¹⁶⁵ (100 ng in 20 ul PBS; R&D Systems) or PBS injected subcutaneously at the same location. Thirty minutes later and a skin circle circumscribing the blue dye was excised, and Evans blue content was determined as above.

e. Bovine Coronary Venule Endothelial Cell (BCVEC) Culture and Monolayer Permeability.

BCVEC (passage 10-15) were seeded onto standard culture dishes or onto 0.4 μm transwell inserts (Corning) (3×10⁵ cells/insert), both precoated with 1.5% gelatin, and maintained in Dulbecco's modified Eagle's medium (DMEM) with 20% fetal bovine serum (FBS) at 10% CO₂ until a tight confluent monolayer was achieved. Cells were then pre-treated with mini-TyrRS for 10 minutes, followed by 100 ng/ml VEGF for 30 minutes. Monolayers were then treated with Evans blue-bovine serum albumin complex (0.67 g/l and 40 g/l) in HEPES-buffered saline for 30 min. Evans blue-albumin in the lower well was measured at 610 nm absorbance. Trans-endothelial albumin flux is expressed as percent clearance of albumin, compared with untreated controls.

f. Thoracic Aorta Organ Culture.

Rat thoracic aortae were isolated and maintained in serum-free medium composed of DMEM/F12, 10 mg/l insulin, 5 μg/l selenium and 5.5 mg/l transferrin in 21% or 1% O₂. After exposure to VEGF for 4 days, samples were frozen in liquid nitrogen for immunoblot assay.

g. Immunohistochemistry.

Confluent BCVEC grown on gelatin-coated glass coverslips were growth-arrested in 0.5% FBS medium for 24 hours. Mini-TyrRS was added 1 hour before 100 ng/ml VEGF treatment. After 1 hour cells were fixed with 2% PFA for 10 minutes, permeabilized in 0.1% Triton X-100 for 5 minutes, and incubated with VE-cadherin primary antibody (1:100, sc-6458, Santa Cruz) and Cy3-conjugated secondary antibody (1:600). Images were digitized at identical settings.

For ear capillary density, ears were perfusion-fixed with 4% PFA in PBS (pH 7.4) at 100 mmHg. Ears were post-fixed in 4% PFA for 24 hours and embedded in paraffin. 8 um thick sections located 5500 um from the distal tip of the pinna were quantified for capillary density after staining for CD31 (sc-1506, 1:50, Santa Cruz), followed by Cy3-conjugated secondary antibody (1:600). Vessels were imaged in 8 different fields (200× magnification) that covered the entire ear (cartilage, skin surface and hair follicles with auto-fluoresence were excluded). Capillary density was derived from mean intensity of CD31 immunofluorescence using Image-J software. T-cells and leukocytes were stained in adjacent sections with rat anti-mouse CD4 antibody (1:50, sc-13573, Santa Cruz) and CD45 antibody (1:200, 30-F11, BD Pharmingen) respectively, followed with Cy3-conjugated secondary antibody (1:400-600). CD4 and CD45 positive cells were counted for the entire ear cross-section at 400× magnification.

h. Immunoblot and Immunoprecipitation.

Tissues and cells were lysed in 1.5% triton-X100 lysis buffer containing protease inhibitors (30 μg/ml aprotinin, 1 mM phenylmethylsulfonyl fluoride, 1 ug/ml leupeptin and 1 ug/ml pepstatin) and phosphatase inhibitors (1 mM sodium-orthovanadate, 2.5 mM sodium-pyrophosphate). Samples were electrophoresed through 10% SDS-polyacrylamide and transferred to nitrocellulose membranes. Membranes were probed with antibodies against mini-TyrRS (1:1000 dilution) and tubulin (1:5000, ab6160, Abcam) followed by Alexa Fluor-680 (Molecular Probes) and IRDye 800 (Rockland) secondary antibodies at 1:5000. Membranes were scanned and analyzed (LI-COR Biosciences).

i. Statistics.

Data are given as means±SEM. Differences were subjected to unpaired t-tests (2-tailed) or ANOVA followed by Bonferroni tests for multiple comparisons (2-tailed). P<0.05 was considered significant.

### Results

a. Mini-TyrRS has Biphasic Effects on Ischemic Angiogenesis

Previous studies have reported that mini-TyrRS induced angiogenesis in cultured endothelial cell, CAM and mouse matrigel assays^{1,2}. To evaluate the in vivo activity of mini-TyrRS in an animal model of ischemia, its effects were examined in a mouse model of ear ischemia produced by ligation of the peripheral and central ear arteries that leaves the proximal-lateral branch of the central artery intact (FIG. 1). In the PBS control group, perfusion declined 50% immediately after ligation, followed by recovery within 3-5 days mediated by angiogenesis and growth of collateral connections among the above arterial trees (FIG. 1A).

Unexpectedly, local subcutaneous injection (20 ul) into the base of the ear of 3 ug/kg/day mini-TyrRS inhibited, while 600 ug/kg/day augmented, recovery of perfusion. Doses of 0.05 and 30 ug/kg/day had no effect (n=8 mice/dose, data not shown), underscoring the biphasic activity. Mutant mini-TyrRS (ELR mutated to EYR) had no effect.

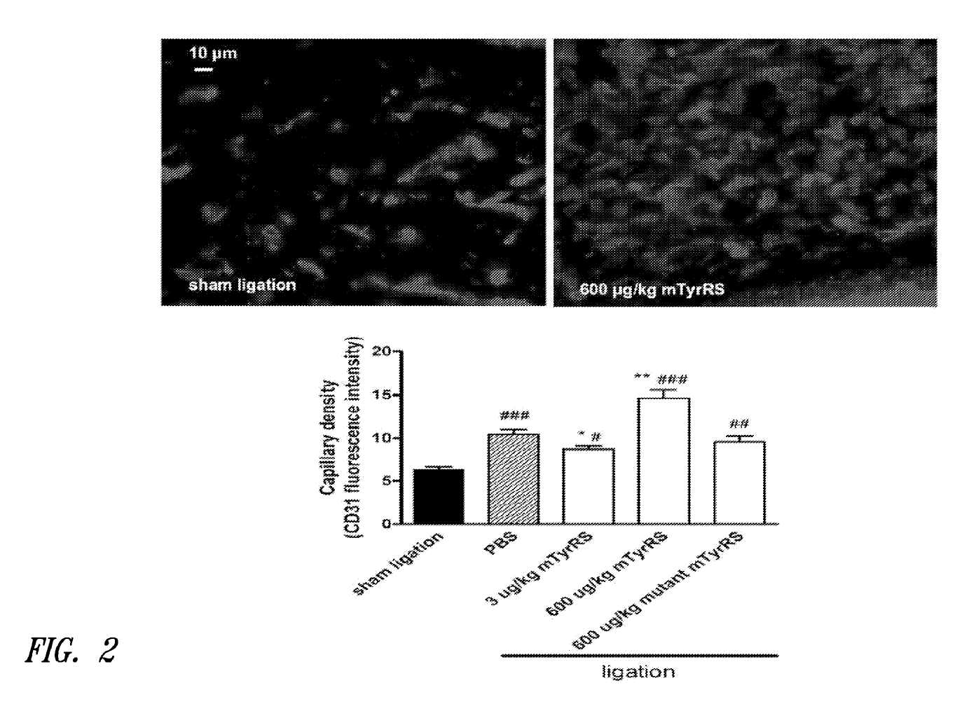

The biphasic effect of mini-TyrRS on recovery of ear perfusion was accompanied by similar changes in capillary density. Capillary density, which increased in ischemia as expected, was inhibited by 3 ug/kg/day and augmented by 600 ug/kg/day mini-TyrRS, whereas mutant mini-TyrRS had no effect (FIG. 2). Body weight did not differ between PBS vehicle or drug groups at any time point (nor in the hindlimb experiment described below). In addition, local subcutaneous injection of mini-TyrRS in the ear was not accompanied by erythema or edema.

Previous in vitro studies had only reported angiogenic activity of miniTyrRS. Therefore, we sought to determine whether systemic administration of mini-TyrRS at a rate (12 ug/kg/h, subcutaneous minipump) targeted to achieve tissue levels similar to that for low-dose in the ear model (local injection), would inhibit recovery of perfusion in a second model of ischemia, i.e., the mouse hindlimb after femoral artery ligation. Mini-TyrRS inhibited recovery of plantar hindpaw perfusion (p=0.016, FIG. 1B), a measure which correlates well with overall hindlimb blood flow⁶. In agreement with reduced recovery of perfusion, ischemic appearance scores were worse in the mini-TyrRS-treated group (FIG. 1C).

b. Mini-TyrRS Increases Leukocyte Accumulation in Ischemia

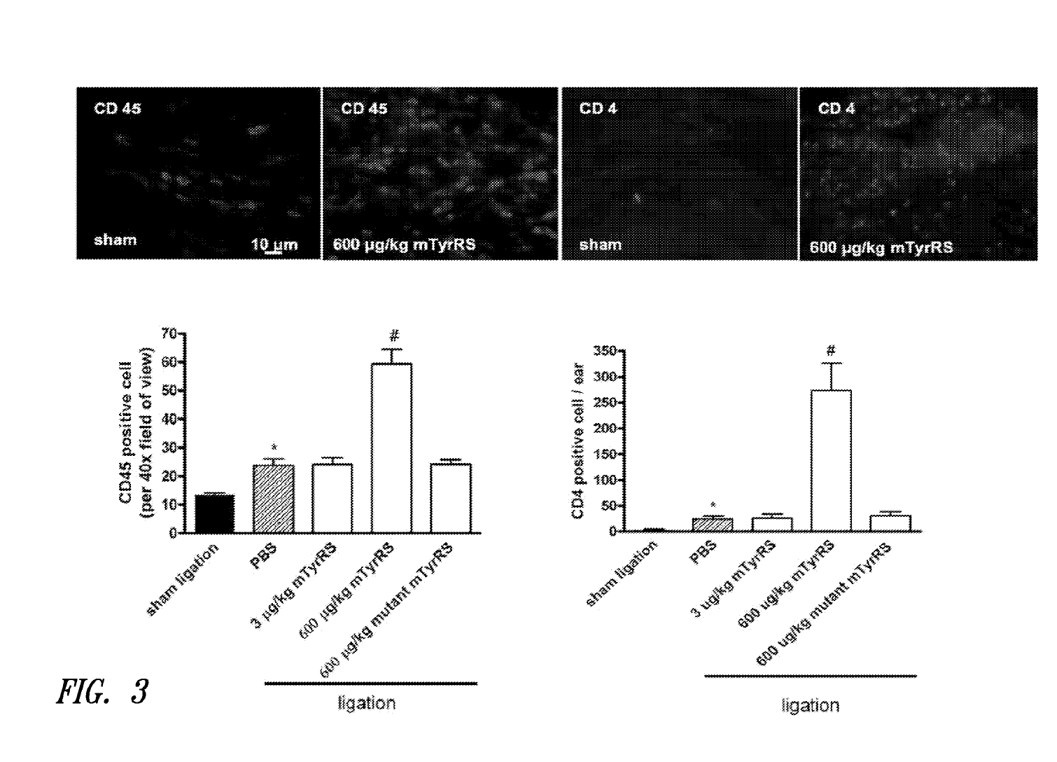

Monocytes/macrophages and T-lymphocytes are involved in angiogenesis^{8,9}. Furthermore, in vitro studies have found that mini-TyrRS stimulates monocyte adhesion and transmigration¹. We therefore examined whether mini-TyrRS affects leukocyte activity in ischemia in cross-sections of the ear adjacent to those used for determining capillary density in FIG. 2. CD45⁺ and CD4⁺ cells increased when measured seven days after ligation (FIG. 3). High-dose but not low-dose mini-TyrRS caused a further increase of both cell types, whereas mutant mini-TyrRS was without effect.

c. Mini-TyrRS has Biphasic Effects on Baseline and Evoked Increase in Permeability

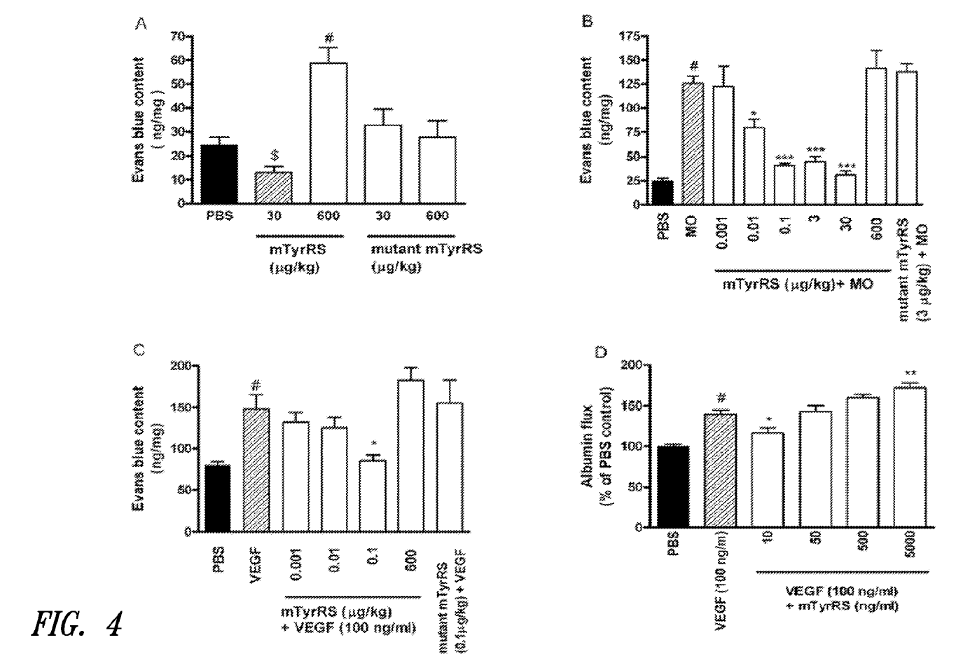

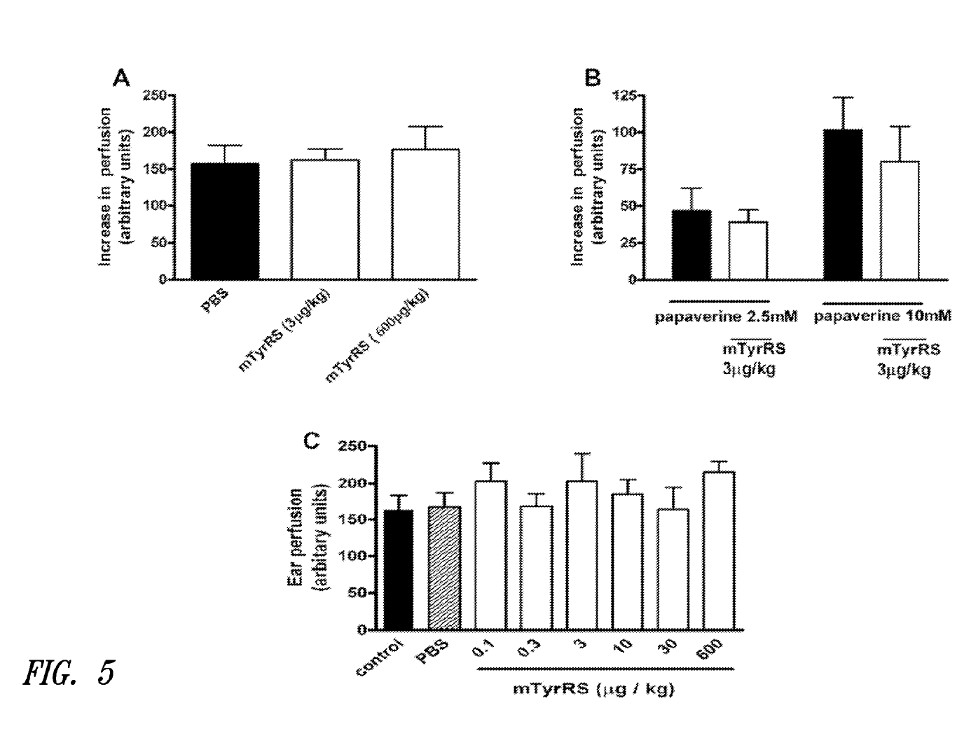

Increased endothelial macromolecular permeability is an important step in the initial phase of angiogenesis, and several angiogenic factors regulate angiogenesis in part through alterations in permeability^{10,11,12}. We thus examined whether mini-TyrRS modulates permeability, using extravasation of Evans blue-conjugated albumen in the normal ear. Similar to its biphasic effects on angiogenesis (FIGS. 1 & 2), mini-TryRS also had biphasic effects on permeability (FIG. 4). Low-dose mini-TyrRS (30 ug/kg) reduced baseline permeability by 50%, while high-dose (600 ug/kg) increased permeability greater than 2-fold (FIG. 4A); mutant mini-TyrRS had no effect. We also tested mini-TyrRS on mustard oil-induced increase in permeability. Mini-TyrRS caused dose-dependent inhibition of induced leakage at low concentrations, with maximal inhibition at 3 ug/kg, whereas 600 ug/kg tended to augment induced permeability (FIG. 4B). Mutant mini-TyrRS had no effect, suggesting, like its actions on angiogenesis, leukocyte accumulation and baseline permeability, that the ELR motif is required for mini-TyrRS's modulation of mineral oil-induced permeability. Similar results were obtained with VEGF-induced leakage (FIG. 4C). The biphasic action of mini-TyrRS on VEGF-induced leakage was confirmed in endothelial cell monolayers (FIG. 4D).

d. Mini-TyrRS Lacks Vasoactive Actions

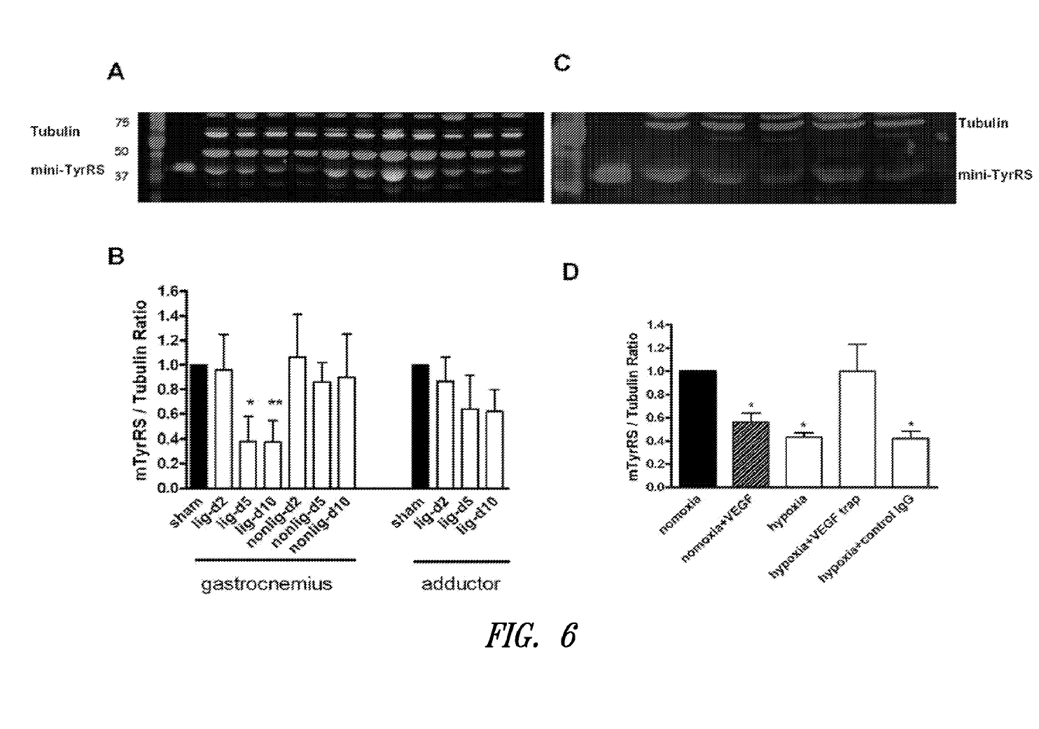

Because some angiogenic factors such as VEGF⁸ exhibit vasoactivity and since such activity of mini-TyrRS could impact recovery of flow in the ear and hindlimb ligation studies (FIG. 1), we evaluated the effect of miniTyrRS on Doppler perfusion of the normal (non-ligated) mouse ear. The following protocols reflect the requirement of ten minutes to obtain the laser scanning Doppler perfusion measurement. In the first experiment, baseline perfusion was measured at 35° C. rectal temperature to assure strong basal vascular tone, followed immediately by local administration of mini-TyrRS or PBS (20 ul here and below) into the base of the ear. Temperature was then raised to 37.5° C. over ten minutes and perfusion was obtained again. Three or 600 ug/kg mini-TyrRS had no effect on temperature-induced increase in perfusion (FIG. 6A). In a second experiment conducted at 35° C., baseline perfusion was obtained, mini-TyrRS or PBS was injected, thirty minutes later the vasodilator papaverine was injected in the same location, and perfusion was obtained again thirty minutes later. Mini-TyrRS had no effect on papavarine-mediated dilation (FIG. 6B). In a third experiment mini-TyrRS was administered locally on six consecutive days. Ear perfusion was measured at 36.5° C. 24 hours after each administration and just before repeat-dosing. Mini-TyrRS had no effect on perfusion on day-six (FIG. 6C) nor at any of the five earlier days (data not shown). Absence of vasoactive effects in these experiments is consistent with absence of erythema noted at any times or dosages, either immediately after or 24 hours after mini-TyrRS administration. Edema was not evident at the 600 ug/kg dosages in any of the experiments, even though this dose increased baseline permeability (FIG. 4). The may reflect low interstitial compliance and/or efficient lymphatic conductance in the ear.

e. Hypoxia and VEGF Downregulate Mini-TyrRS

Angiogenesis in response to tissue hypoxia and ischemia is achieved through upregulation of angiogenic factors such as VEGF, which in turn or through other mechanisms, downregulate angiostatic factors⁸. If endogenous mini-TyrRS normally exerts angiostatic actions at physiological concentrations, as indicated by the above findings with low-dose mini-TyrRS for recovery of flow, angiogenesis and permeability, then tissue levels of mini-TyrRS might be regulated negatively in ischemia and in response to VEGF. Mini-TyrRS levels were examined by immunoblot in muscle that experiences strong (gastrocnemius) versus little or no ischemia (adductor) after femoral artery ligation¹³. Mini-TyrRS decreased in the gastrocnemius but not adductor of the ligated leg, when compared to the gastrocnemius from sham animals (no surgery) or from the contralateral non-ligated leg (FIGS. 7A & 7B). To test possible involvement of hypoxia and VEGF in this downregulation, we examined rat thoracic aorta maintained in organ culture. Four days of exposure to VEGF or hypoxia (1% O₂) caused similar downregulation of mini-TyrRS (FIGS. 7C & 7D). Moreover, hypoxic downregulation was abolished by VEGF neutralizing trap. These data suggest that hypoxic induction of VEGF may mediate downregulation of mini-TyrRS in ischemic tissue.

### DISCUSSION

We have evaluated potential actions of mini-TyrRS on ischemic angiogenesis, leukocyte trafficking, permeability and vasoactivity in vivo, and have identified unexpected biphasic actions on angiogenesis and permeability.

In the mouse ear model of ischemia, low-dose mini-TyrRS (3 ug/kg/day) inhibited while high-dose (600 ug/kg/day) augmented angiogenesis (doses were given as two injections twelve hours apart). Low-dose mini-TyrRS (12 ug/kg/h) also reduced recovery of perfusion in the mouse hindlimb ligation model. In contrast to this angiostatic-like action at low dose, in a previous study only angiogenic actions were observed², i.e., mini-TyrRS was angiogenic at 2.4-24 ug/ml (60-600 nmol/L) in matrigel explants and induced migration of cultured endothelial cells at 2 ug/ml (50 nmol/L).² This discrepancy could arise for several reasons, including the context of ischemia in our study, inherent differences in conditions in vivo, in vitro and “in matrigel”, and because the concentrations used previously² are undoubtedly higher than achieved in our low-dose groups, considering the effects of dilution and degradation over time. For example, assuming mini-TyrRS injected locally into the ear distributes into the extracellular space and that degradation reduces the concentration by at least 10-fold, our low- and high-dose regimens would achieve average extracellular concentrations of 0.006 ug/ml and 1.2 ug/ml. Thus, although concentrations in the ear's extracellular fluid would clearly be higher for some duration after injection, it is likely that time-averaged levels achieved in our low-dose groups were significantly lower that in previous studies.

Recruitment of leukocytes and endothelial progenitor cells (EPCs) contributes importantly to angiogenesis in ischemia, inflammation and tumor growth⁴. These recruited cells exhibit heterogeneous phenotypes, expressing markers for macrophages, T-cells, SMCs, fibroblasts, pericytes and EPCs, and secrete growth factors and cytokines which act directly or indirectly to augment endothelial cell migration, proliferation and capillary sprouting. CD4-positive T-cells are a major subgroup of T-lymphocytes that play an important role in angiogenesis by secreting angiogenic growth factors such as VEGF¹⁴ and bFGF¹⁵. In the present study, high-dose mini-TyrRS increased leukocyte (CD45-positive) accumulation in the ischemic ear, especially CD4-positive cells, by about 10-fold, while low-dose mini-TyrRS had no effect. This action may contribute to the angiogenic effect of mini-TyrRS.

Besides the biphasic effects of mini-TyrRS on angiogenesis, another unique finding is that mini-TyrRS also has biphasic effects on both basal and evoked permeability. In most circulations including the cutaneous vasculature, permeability to plasma proteins and smaller molecules is normally low. Ischemia, inflammation and tumor growth are accompanied by increased vascular permeability which is an important early step in angiogenesis in these conditions¹⁶. The resulting leakage of plasma proteins and other circulating macromolecules helps to convert the normally anti-angiogenic stroma into a strongly pro-angiogenic provisional stroma^{8,17}. Many angiogenic factors such as VEGF¹⁶, bFGF¹⁰, interleukin-8¹¹, angiopoietin-2¹² and thrombin^{18,19} increase endothelial permeability. It has also been recently shown that increased permeability is critical for the angiogenic effects of EPCs and bone marrow-derived mononuclear cells²⁰. On the other hand, antagonism of increased permeability reduces angiogenesis,^{21,22} and angiostatic proteins such as angiostatin²³, caveolin-1²¹ and TNP-470²² strongly reduce permeability. However to our knowledge, no endogenous angiostatic factor has been reported to reduce basal permeability like that observed in the present study for mini-TyrRS. We also found that mini-TyrRS at low doses caused dose-dependent inhibition of leakage induced by mustard oil and VEGF, while high-dose mini-TyrRS tended to augment evoked leakage. Normals baseline permeability displayed similar biphasic regulation by mini-TyrRS. These biphasic effects on basal and evoked permeability may contribute to the angiogenic action of mini-TyrRS. Although the mechanisms underlying both of these biphasic effects await future studies, estrogen has similar biphasic effects on permeability in EC in vitro²⁴. The specificity of our findings regarding angiogenesis, leukocyte accumulation and permeability are supported by their dependence on an intact ELR motif. This motif is known to be required for receptor binding, neutrophil activation and angiogenesis induced by mini-TyrRS in vitro^{1,2} and other ELR-containing chemokines such as Interleukin-8²⁵.

Tissue hypoxia in ischemia and tumor growth strongly induces many of the steps involved in angiogenesis, eg, increased permeability²⁶, inflammation^{27,28}, endothelial cell proliferation and migration,²⁹ and matrix degradation³⁰. Many of the genes that mediate these processes, such as eNOS³¹, VEGF²⁶, angiopoietin-2³², AKT³³ and bFGF³⁴, are regulated by hypoxia, generally through the presence of hypoxia regulated element sequences in the 5′ and mRNA stabilizing sequences in the 3′ regions. Expression of mini-TyrRS was significantly down-regulated in gastrocnemius muscle when examined at 5 and 10 days after ligation of the femoral artery. Expression was similarly reduced in thoracic aorta by either hypoxia or VEGF in vitro. Moreover, blockade of endogenous VEGF abolished hypoxic down-regulation of mini-TyrRS in this in vitro model. These findings suggest that the reduction of mini-TyrRS in hindlimb muscle after femoral ligation is mediated by VEGF.

The anti-angiogenic and anti-permeability actions of miniTyrRS, together with its down-regulation in ischemia, have interesting similarities to the atypical angiogenic protein, angiopoietin-1. Angiopoietin-1 inhibits permeability, is angiostatic under certain conditions³⁵, and is down-regulated after ligation^{7,36}. Angiopoietin-1 also has angiogenic actions under certain conditions³⁷. To our knowledge, mini-TyrRS is the first factor observed to inhibit angiogenesis at low and stimulate it at high concentrations.

In summary, we have demonstrated that low-dose mini-TyrRS inhibits basal and evoked permeability and ischemic angiogenesis. On the other hand, high-dose has opposite effects and, in addition, augments recruitment of CD45-positive and CD4-positive cells in ischemic tissue. Mechanistically, we have shown that the inhibitory effects of mini-TyrRS occur at concentrations that inhibit E-cadherin translocation, and provide evidence that mini-TyrRS is down-regulated by VEGF-dependent signaling in ischemia. These findings indicate that low-dose administration of mini-TyrRS in vivo can be used to achieve angiostatic effects.

From the foregoing, it will be appreciated that, although specific embodiments of the invention have been described herein for the purpose of illustration, various modifications may be made without deviating from the spirit and scope of the invention.

### REFERENCES

1. Wakasugi K, Schimmel P. Two distinct cytokines released from a human aminoacyl-tRNA synthetase. Science. 1999; 284:147-51.
2. Wakasugi K, Slike B M, Hood J, Ewalt K L, Cheresh D A, Schimmel P. Induction of angiogenesis by a fragment of human tyrosyl-tRNA synthetase. J Biol Chem. 2002; 277:20124-6.
3. Wakasugi K, Slike B M, Hood J, Otani A, Ewalt K L, Friedlander M, Cheresh D A, Schimmel P. A human aminoacyl-tRNA synthetase as a regulator of angiogenesis. Proc Natl Acad Sci USA. 2002; 99:173-7.
4. Frantz S, Vincent K A, Feron O, Kelly R A. Innate immunity and angiogenesis. Circ Res. 2005; 96:15-26.
5. Ahn S T, Mustoe T A. Effects of ischemia on ulcer wound healing: a new model in the rabbit ear. Ann Plast Surg. 1990; 24:17-23.
6. Chalothorn D, Clayton J A, Zhang H, Pomp D, Faber J E. Collateral density, remodeling, and VEGF-A expression differ widely between mouse strains. Physiol Genomics. 2007; 30:179-91.
7. Thurston G, Suri C, Smith K, McClain J, Sato T N, Yancopoulos G D, McDonald D M. Leakage-resistant blood vessels in mice transgenically overexpressing angiopoietin-1. Science. 1999; 286:2511-4.
8. Carmeliet P. Angiogenesis in health and disease. Nat Med. 2003; 9:653-60.
9. Heil M, Eitenmuller I, Schmitz-Rixen T, Schaper W. Arteriogenesis versus angiogenesis: similarities and differences. J Cell Mol Med. 2006; 10:45-55.
10. Cao R, Eriksson A, Kubo H, Alitalo K, Cao Y, Thyberg J. Comparative evaluation of FGF-2-, VEGF-A-, and VEGF-C-induced angiogenesis, lymphangiogenesis, vascular fenestrations, and permeability. Circ Res. 2004; 94:664-70.
11. Rampart M, Van Damme J, Zonnekeyn L, Herman A G. Granulocyte chemotactic protein/interleukin-8 induces plasma leakage and neutrophil accumulation in rabbit skin. Am J Pathol. 1989; 135:21-5.
12. Roviezzo F, Tsigkos S, Kotanidou A, Bucci M, Brancaleone V, Cirino G, Papapetropoulos A. Angiopoietin-2 causes inflammation in vivo by promoting vascular leakage. J Pharmacol Exp Ther. 2005; 314:738-44.
13. Ito W D, Arras M, Scholz D, Winkler B, Htun P, Schaper W. Angiogenesis but not collateral growth is associated with ischemia after femoral artery occlusion. Am J Physiol. 1997; 273:H1255-65.
14. Freeman M R, Schneck F X, Gagnon M L, Corless C, Soker S, Niknejad K, Peoples G E, Klagsbrun M. Peripheral blood T lymphocytes and lymphocytes infiltrating human cancers express vascular endothelial growth factor: a potential role for T cells in angiogenesis. Cancer Res. 1995; 55:4140-5.
15. Blotnick S, Peoples G E, Freeman M R, Eberlein T J, Klagsbrun M. T lymphocytes synthesize and export heparin-binding epidermal growth factor-like growth factor and basic fibroblast growth factor, mitogens for vascular cells and fibroblasts: differential production and release by CD4+ and CD8+ T cells. Proc Natl Acad Sci USA. 1994; 91:2890-94.
16. Bates D O, Harper S J. Regulation of vascular permeability by vascular endothelial growth factors. Vascul Pharmacol. 2002; 39:225-37.
17. Dvorak H F, Senger D R, Dvorak A M, Harvey V S, McDonagh J. Regulation of extravascular coagulation by microvascular permeability. Science. 1985; 227:1059-61.
18. Rabiet M J, Plantier J L, Rival Y, Genoux Y, Lampugnani M G, Dejana E. Thrombin-induced increase in endothelial permeability is associated with changes in cell-to-cell junction organization. Arterioscler Thromb Vasc Biol. 1996; 16:488-96.
19. Killackey J J, Johnston M G, Movat H Z. Increased permeability of microcarrier-cultured endothelial monolayers in response to histamine and thrombin. A model for the in vitro study of increased vasopermeability. Am J Pathol. 1986; 122:50-61.
20. You D, Waeckel L, Ebrahimian T G, Blanc-Brude O, Foubert P, Barateau V, Duriez M, Lericousse-Roussanne S, Vilar J, Dejana E, Tobelem G, Levy B I, Silvestre J S. Increase in vascular permeability and vasodilation are critical for proangiogenic effects of stem cell therapy. Circulation. 2006; 114:328-38.
21. Bucci M, Gratton J P, Rudic R D, Acevedo L, Roviezzo F, Cirino G, Sessa W C. In vivo delivery of the caveolin-1 scaffolding domain inhibits nitric oxide synthesis and reduces inflammation. Nat Med. 2000; 6:1362-7.
22. Satchi-Fainaro R, Mamluk R, Wang L, Short S M, Nagy J A, Feng D, Dvorak A M, Dvorak H F, Puder M, Mukhopadhyay D, Folkman J. Inhibition of vessel permeability by TNP-470 and its polymer conjugate, caplostatin. Cancer Cell. 2005; 7:251-61.
23. Sima J, Zhang S X, Shao C, Fant J, Ma J X. The effect of angiostatin on vascular leakage and VEGF expression in rat retina. FEBS Lett. 2004; 564:19-23.
24. Ye L, Martin T A, Parr C, Harrison G M, Mansel R E, Jiang W G. Biphasic effects of 17-beta-estradiol on expression of occludin and transendothelial resistance and paracellular permeability in human vascular endothelial cells. J Cell Physiol. 2003; 196:362-9.
25. Hebert C A, Vitangcol R V, Baker J B. Scanning mutagenesis of interleukin-8 identifies a cluster of residues required for receptor binding. J Biol Chem. 1991; 266:18989-94.
26. Fischer S, Clauss M, Wiesnet M, Renz D, Schaper W, Karliczek G F. Hypoxia induces permeability in brain microvessel endothelial cells via VEGF and NO. Am J Physiol. 1999; 276:C812-20.
27. Demasi M, Cleland L G, Cook-Johnson R J, James M J. Effects of hypoxia on the expression and activity of cyclooxygenase 2 in fibroblast-like synoviocytes: interactions with monocyte-derived soluble mediators. Arthritis Rheum. 2004; 50:2441-9.
28. Zampetaki A, Mitsialis S A, Pfeilschifter J, Kourembanas S. Hypoxia induces macrophage inflammatory protein-2 (MIP-2) gene expression in murine macrophages via NF-kappaB: the prominent role of p42/p44 and PI3 kinase pathways. Faseb J. 2004; 18:1090-2.
29. Yamakawa M, Liu L X, Date T, Belanger A J, Vincent K A, Akita G Y, Kuriyama T, Cheng S H, Gregory R J, Jiang C. Hypoxia-inducible factor-1 mediates activation of cultured vascular endothelial cells by inducing multiple angiogenic factors. Circ Res. 2003; 93:664-73.
30. Ben-Yosef Y, Lahat N, Shapiro S, Bitterman H, Miller A. Regulation of endothelial matrix metalloproteinase-2 by hypoxia/reoxygenation. Circ Res. 2002; 90:784-91.
31. Hoffmann A, Gloe T, Pohl U. Hypoxia-induced upregulation of eNOS gene expression is redox-sensitive: a comparison between hypoxia and inhibitors of cell metabolism. J Cell Physiol. 2001; 188:33-44.
32. Pichiule P, Chavez J C, LaManna J C. Hypoxic regulation of angiopoietin-2 expression in endothelial cells. J Biol Chem. 2004; 279:12171-80.
33. Li W, Petrimpol M, Molle K D, Hall M N, Battegay E J, Humar R. Hypoxia-induced endothelial proliferation requires both mTORC1 and mTORC2. Circ Res. 2007; 100:79-87.
34. Galvani M, Rapisarda A, Uranchimeg B, Shoemaker R H, Melillo G. Hypoxic induction of an HIF-1alpha-dependent bFGF autocrine loop drives angiogenesis in human endothelial cells. Blood. 2006; 107:2705-12.
35. Stoeltzing O, Ahmad S A, Liu W, McCarty M F, Wey J S, Parikh A A, Fan F, Reinmuth N, Kawaguchi M, Bucana C D, Ellis L M. Angiopoietin-1 inhibits vascular permeability, angiogenesis, and growth of hepatic colon cancer tumors. Cancer Res. 2003; 63:3370-7.
36. Sandhu R, Teichert-Kuliszewska K, Nag S, Proteau G, Robb M J, Campbell Al, Kuliszewski M A, Kutryk M J, Stewart D J. Reciprocal regulation of angiopoietin-1 and angiopoietin-2 following myocardial infarction in the rat. Cardiovasc Res. 2004; 64:115-24.
37. Morisada T, Kubota Y, Urano T, Suda T, Oike Y. Angiopoietins and angiopoietin-like proteins in angiogenesis. Endothelium. 2006; 13:71-9.

We claim:

1. A composition comprising a physiologically-acceptable excipient and an angiostatically-effective concentration of a truncated tyrosyl-tRNA synthetase polypeptide.

2. The composition of claim 1 where the truncated tyrosyl-tRNA synthetase comprises a mammalian tyrosyl-tRNA synthetase truncated at its C-terminus.

3. The composition of claim 1 where the truncated tyrosyl-tRNA synthetase comprises a tyrosyl-tRNA synthetase having at least 90% identity to the human tyrosyl-tRNA synthetase of SEQ ID NO: 1 and which is truncated at its C-terminus.

4. The composition of claim 1 where the truncated tyrosyl-tRNA synthetase comprises the human tyrosyl-tRNA synthetase of SEQ ID NO: 1 truncated at its C-terminus.

5. The composition of claim 1 where the truncated tyrosyl-tRNA synthetase comprises a full length tyrosyl-tRNA synthetase having about 50-100 amino acid residues truncated from its C-terminus.

6. The composition of claim 1 where the truncated tyrosyl-tRNA synthetase comprises a full length tyrosyl-tRNA synthetase having about 100-150 amino acid residues truncated from its C-terminus.

7. The composition of claim 1 where the truncated tyrosyl-tRNA synthetase comprises a full length tyrosyl-tRNA synthetase having about 150-200 residues truncated from its C-terminus.

8. The composition of claim 1 where the truncated tyrosyl-tRNA synthetase comprises a full length tyrosyl-tRNA synthetase having about 200-250 amino acid residues truncated from its C-terminus.

9. The composition of claim 1 where the truncated tyrosyl-tRNA synthetase comprises the sequence ELR.

10. The composition of claim 1 where the truncated tyrosyl-tRNA synthetase comprises a Rossmann fold nucleotide binding domain.

11. The composition of claim 1 where the truncated tyrosyl-tRNA synthetase consists essentially of amino acid residues 1-364 or 1-343 of SEQ ID NO: 1.

12. The composition of claim 1 where the angiostatically-effective concentration is a concentration ranging from about 1-10 ug/kg.

13. A method for treating a condition that would benefit from decreased angiogenesis comprising administering to a subject a composition comprising a physiologically-acceptable excipient and an angiostatically-effective concentration of a truncated tyrosyl-tRNA synthetase.

14. The method of claim 13 where the condition to be treated is selected from the group consisting of cancer (including solid and hematological tumors), rheumatoid arthritis, other arthritides, psoriasis, hyperangiogenic diseases, diabetic retinopathy, retinopathy of prematurity, ischemic retinopathy, macular degeneration, diabetic nephropathy, and sepsis.

15. The method of claim 13 where the truncated tyrosyl-tRNA synthetase comprises a mammalian tyrosyl-tRNA synthetase truncated at its C-terminus.

16. The method of claim 13 where the truncated tyrosyl-tRNA synthetase comprises a tyrosyl-tRNA synthetase having at least 90% identity to the human tyrosyl-tRNA synthetase of SEQ ID NO: 1 and which is truncated at its C-terminus.

17. The method of claim 13 where the truncated tyrosyl-tRNA synthetase comprises the human tyrosyl-tRNA synthetase of SEQ ID NO: 1 truncated at its C-terminus.

18. The method of claim 13 where the truncated tyrosyl-tRNA synthetase comprises a full length tyrosyl-tRNA synthetase having about 50-100 amino acid residues truncated from its C-terminus.

19. The method of claim 13 where the truncated tyrosyl-tRNA synthetase comprises a full length tyrosyl-tRNA synthetase having about 100-150 amino acid residues truncated from its C-terminus.

20. The method of claim 13 where the truncated tyrosyl-tRNA synthetase comprises a full length tyrosyl-tRNA synthetase having about 150-200 residues truncated from its C-terminus.

21. The method of claim 13 where the truncated tyrosyl-tRNA synthetase comprises a full length tyrosyl-tRNA synthetase having about 200-250 amino acid residues truncated from its C-terminus.

22. The method of claim 13 where the truncated tyrosyl-tRNA synthetase comprises the sequence ELR.

23. The method of claim 13 where the truncated tyrosyl-tRNA synthetase comprises a Rossmann fold nucleotide binding domain.

24. The method of claim 13 where the truncated tyrosyl-tRNA synthetase consists essentially of amino acid residues 1-364 or 1-343 of SEQ ID NO: 1.

25. The method of claim 13 where the angiostatically-effective concentration is a concentration ranging from about 1-10 ug/kg.
